(19) United States
(12) Patent Application Publication
(10) Pub. No.: US 2023/0342921 A1
(43) Pub. Date: Oct. 26, 2023

(54) METHODS AND RELATED ASPECTS FOR MEDICAL IMAGE GENERATION

(71) Applicant: THE JOHNS HOPKINS UNIVERSITY, Baltimore, MD (US)

(72) Inventors: Yong DU, Lutherville-Timonium, MD (US); Kevin H. LEUNG, Baltimore, MD (US); Martin Gilbert POMPER, Baltimore, MD (US); Steven P. ROWE, Baldwin, MD (US); Wenyi SHAO, Laurel, MD (US)

(73) Assignee: THE JOHNS HOPKINS UNIVERSITY, Baltimore, MD (US)

(21) Appl. No.: 18/004,941

(22) PCT Filed: Jul. 21, 2021
(86) PCT No.: PCT/US2021/042656

§ 371 Date: Jan. 10, 2023

Related U.S. Application Data
(60) Provisional application No. 63/055,324, filed on Jul. 22, 2020.

(51) Int. Cl. G06T 7/00; G16H 30/40; G16H 50/50

(52) U.S. Cl. CPC G06T 7/0012; G16H 30/40; G16H 50/50; G06T 2207/10081; G06T 2207/10088; G06T 2207/10104; G06T 2207/10108; G06T 2207/20084; G06T 2207/30016

(57) ABSTRACT

Provided herein are methods of generating medical images that include the use of a generative adversarial network (GAN) in certain embodiments. Related methods, systems, and computer program products are also provided.

### CROSS-REFERENCE TO RELATED APPLICATIONS

This application claims priority to U.S. Provisional Patent Application Ser. No. 63/055,324, filed Jul. 22, 2020, the disclosure of which is incorporated herein by reference.

### STATEMENT OF GOVERNMENT SUPPORT

This invention was made using U.S. Government support under NIH Grant No. NS094227. The U.S. Government has certain rights in this invention.

### BACKGROUND

In quantitative single photon emission computed tomography (SPECT) and positron emission tomography (PET) imaging, Monte Carlo simulation is an indispensable tool for the assessment of reconstruction algorithms, compensation methods, quantification and data analysis methods. In a simulation study, the ground truth is known and the imaging physics can be accurately modeled with Monte Carlo simulation. Thus, using simulated data can provide an accurate measurement of the accuracy and precision. Simulation studies are widely used for task-based image quality assessment studies. They are also often used for the initial validation and optimization of new camera system designs to reduce cost. Compared to physical phantom experiments, which can only provide limited numbers of rigid organ components, simulations with realistic digital phantoms can accurately mimic human anatomy and variation. Compared to clinical data, truth is known and thus an unbiased assessment is achievable.

Recently, machine learning (ML)-based techniques have been successfully implemented in MRI, CT, and PET image reconstruction and have been shown to produce higher-quality images with lower noise, higher contrast, and higher resolution than conventional approaches. ML techniques have also been applied in image segmentation, assessing prognosis, and assisting diagnosis, among other applications. However, most of those applications used only a small dataset (tens to hundreds), which could potentially cause overfitting due to the limited data size, or underfitting if the variation in the data is small. In addition, clinical data was often used in training. Since the ground truth is unknown for clinical data, a trained deep neural network may produce suboptimal results. For example, in ML-based reconstruction of PET or SPECT, the ordered subset expectation maximization (OS-EM) reconstructed images are typically used as ground truth to train a network to restore filtered back projection (FBP) reconstructed images. This results in an image with somewhat improved image quality that merely matches the quality of OS-EM reconstruction, which is already widely available in clinical systems. This could undermine the impact of ML-based methods. To realize the full potential of machine learning in SPECT and PET imaging, a large number of simulated images and data with known ground truth should be used to train the artificial neural network.

There have been various attempts to use ML-based methods to generate synthetic SPECT or PET images for various research purposes. Those synthetic images, while visually similar to a real clinical SPECT or PET image, lack any accurate modeling of true physical effects. Such physical effects, such as scatter and spatial resolution, are shift variant and highly depend on not only reconstruction algorithms and parameters, but also the patient specific anatomy and activity distribution. Most importantly, true activity uptake and anatomical information are still unknown in those synthetic SPECT and PET images. These factors make it impossible to use those synthesized images for research purposes, such as validating reconstruction algorithms, evaluating quantitative accuracy, and training other neural networks on other tasks which require known ground truth.

Overall, simulation is still a widely used tool for modern day SPECT and PET research. A large set of digital phantoms that realistically mimic the variations in anatomy and tracer distribution of the human population is typically needed to simulate what can be encountered in real human studies. However, most of the currently available digital phantoms, such as the Zubal phantom and the XCAT phantom, are usually developed based on averaged or mean human data. They lack the ability to produce the variations in organ size and shape and body size and shape that can be seen in clinics. One solution is to use anatomical imaging data, such as CT or MRI, to produce a phantom population. For example, in brain SPECT and PET studies, MRI images can be used to obtain anatomical information with high spatial resolution and high contrast for soft tissues. However, for task-based image quality assessments and ML-based research, thousands of images are often needed. It is difficult to obtain such a large amount of clinical MRI data. Additionally, the time required for processing the data for phantom generation is typically prohibitive.

Recently, generative adversarial networks (GANs) have been used to generate clinically realistic 2D MRI images. However, a GAN-based approach to generate realistic synthetic 3D brain structural MRI images that reflect the anatomical variability of both the normal and abnormal patient populations had previously not been achieved. Unlike the synthetic SPECT and PET images referenced above, those synthetic brain MRI images would be able to provide accurate 3D anatomical information. Such anatomical information could be used as phantoms for generating realistic activity distributions and attenuation maps needed for SPECT and PET simulations. The 3D anatomical information from the synthetic brain MRI images could also be used as region-of-interest maps for quantitative analysis. Further, such a method could be used to generate an unlimited number of phantoms as needed with adequate anatomical variability as seen in clinic. This would provide a large dataset that is not limited by lack of clinical data for SPECT and PET research.

Accordingly, there is a need for additional ML-based analytical tools, methods, and related aspects, for generating medical images of use in diagnosing and/or prognosticating various pathologies.

### SUMMARY

The present disclosure relates, in certain aspects, to methods, systems, and computer readable media of use in producing a large digital phantom population that reflects the anatomical variability of both normal and abnormal patient populations. In other aspects, the present disclosure provides for the use of an image compression technique based on an autoencoder network. This dimensionality reduction technique can compress the image dimensions and thus make the task of training a GAN-based approach to generate synthetic 3D images more efficient. The techniques disclosed herein can change the conventional ways of training learnable systems for medical image applications. In some aspects, for example, the methods disclosed herein include training a convolutional neural network (CNN) to produce SPECT or PET activity distributions and attenuation maps directly from MRI images. Also provided are methods of training a cycle consistent generative adversarial network (CycleGAN) to produce synthetic SPECT or PET activity distributions and attenuation maps from a distribution of MRI images in certain embodiments. These methods can be directly applied to real patient MRI images. Methods of validating the anatomical realism and accuracy of the synthetic MRI images using existing brain MRI segmentation and analysis software is also disclosed herein and can provide for a quantitative evaluation of the realism of the synthetized images. These and other aspects will be apparent upon a complete review of the present disclosure, including the accompanying figures.

In some embodiments, GANs are trained to generate synthetic MRI images before converting them to the activity distributions and attenuation maps for simulation. One exemplary alternative disclosed herein is to directly generate activity distributions and attenuation maps using the GANs. In some of these applications, however, true data is unavailable to train such GANs. The true data here refers to images with known tracer distributions and accurate anatomical information. Existing patient data, such as SPECT images and PET images, are often corrupted by limited spatial resolution, scatter, partial volume effects, and noise. Thus, they typically cannot be used to train a GAN without introducing bias. On the other hand, MRI images often have very high spatial resolution and signal to noise ratio. They contain accurate anatomical information that can be used for generating accurate phantoms. In fact, most of anthropomorphic digital phantoms used in SPECT and PET simulation studies, such as Zubal and XCAT, are constructed based on high resolution MRI images or CT images. Therefore, in certain embodiments, GANs are initially trained to produce synthetic MRI images with accurate anatomical information, which images are then converted to activity distributions and attenuation maps. The data generated from such synthetic MRI images can, for example, be used to train a GAN to directly produce activity distribution and attenuation maps in certain applications.

In certain embodiments, the methods disclosed herein are used for generating a brain phantom population that can be used for simulating brain SPECT and PET images for various neurological disorders. The simulated data can also be used for investigating reconstruction, compensation, and data analysis methods with known ground truth. It can also be used to train other ML-based methods. The methods disclosed herein are also optionally used to generate phantom populations of the torso, abdomen, and extremities for simulating cardiac and tumor imaging, among other medical imaging applications.

In one aspect, the present disclosure provides a method of producing at least one activity distribution and/or at least one attenuation map, the method comprising training at least one generative adversarial network (GAN) with data from a plurality of real and/or synthetic magnetic resonance (MR) images and/or data from a plurality of real and/or synthetic computed tomography (CT) images to produce the activity distribution and/or the attenuation map.

In one aspect, the present disclosure provides a method of conducting a medical imaging simulation. The method includes training at least one generative adversarial network (GAN) with data from a plurality of synthetic magnetic resonance (MR) images and/or data from a plurality of synthetic computed tomography (CT) images to produce at least one activity distribution and/or at least one attenuation map. The method also includes using the activity distribution and/or the attenuation map to conduct the medical imaging simulation.

In some aspects, the present disclosure provides a method of producing synthetic magnetic resonance (MR) images and/or synthetic computed tomography (CT) images, the method comprising training at least one generative adversarial network (GAN) with non-mean data from a plurality of real magnetic resonance (MR) images and/or non-mean data from a plurality of real computed tomography (CT) images to produce a plurality of synthetic MR images and/or a plurality of synthetic CT images.

In other aspects, the present disclosure provides a method of producing at least one activity distribution and/or at least one attenuation map. The method includes training at least one generative adversarial network (GAN) with data from a plurality of real magnetic resonance (MR) images and/or data from a plurality of real computed tomography (CT) images to produce a plurality of synthetic MR images and/or a plurality of synthetic CT images that comprises substantially accurate anatomical information. The method also includes converting the plurality of synthetic MR images and/or the plurality of synthetic CT images to the activity distribution and/or the attenuation map.

In some embodiments, the methods disclosed herein include using the activity distribution and/or the attenuation map to conduct one or more medical imaging simulations. In certain embodiments, the imaging simulations comprise a known ground truth. In certain embodiments, the methods disclosed herein include using the medical imaging simulations to perform at least one image-based task. In some embodiments, the medical imaging simulations comprise single photon emission computed tomography (SPECT) and/or positron emission tomography (PET). In certain embodiments, images in the plurality of real MR images and/or the plurality of real CT images are of at least one anatomical part of a plurality of reference subjects.

In some embodiments, the plurality of synthetic MR images and/or the plurality of synthetic CT images comprises a substantially normal distribution of anatomical variability present in the plurality of reference subjects. In certain embodiments, the plurality of reference subjects comprises normal and/or abnormal subjects. In some embodiments, the plurality of synthetic MR images and/or the plurality of synthetic CT images comprises substantially accurate anatomical information. In some embodiments, the data from the plurality of synthetic MR images and/or the data from the plurality of synthetic CT images comprises non-mean data.

In some embodiments, the methods disclosed herein include using the substantially accurate anatomical information to produce at least one regions-of-interest map. In certain embodiments, the anatomical part is selected from the group consisting of: an organ, a torso, an abdomen, an extremity, and a tumor. In some embodiments, the activity distribution and/or the attenuation map models at least one normal population and/or at least one diseased population. In some embodiments, the organ comprises a brain.

In some embodiments, the methods disclosed herein include using at least one compression technique to compress dimensionality of one or more of the plurality of real and/or synthetic MR images and/or the plurality of real and/or synthetic CT images. In some embodiments, the compression technique comprises using at least one autoencoder network. In certain embodiments, the autoencoder network comprises at least one encoder that maps input MR images and/or input CT images to at least one feature map that comprises a lower dimensional space than the input MR images and/or the input CT images to produce compressed images. In some embodiments, the autoencoder network comprises at least one decoder that maps the compressed images to the input MR images and/or to the input CT images. In certain embodiments, the methods disclosed herein include training the autoencoder network using an unsupervised training technique. In some embodiments, the compression technique does not use down-sampling. In certain embodiments, the autoencoder network comprises a stacked configuration comprising multiple autoencoders. In certain embodiments, the autoencoder network comprises using one or more hyperparameters. In some embodiments, the methods disclosed herein include using at least one training procedure that comprises a mean square error (MSE) as a cost function and/or a sparsity regularizer. In some embodiments, the autoencoder network comprises a multi-layer convolutional neural network (CNN) configuration that compresses and abstracts a given image being processed two or more times.

In some embodiments, the methods disclosed herein include using at least one convolutional neural network (CNN) and/or at least one cycle consistent GAN (CycleGAN) to produce the activity distribution and/or the attenuation map. In certain embodiments, the methods disclosed herein include validating anatomical realism and/or accuracy of one or more of the plurality of synthetic MR images, the plurality of synthetic CT images, the activity distribution, and/or the attenuation map. In some embodiments, the methods disclosed herein include quantifying one or more activity distributions in one or more single photon emission computed tomography (SPECT) and/or positron emission tomography (PET) images to convert the plurality of synthetic MR images and/or the plurality of synthetic CT images to the activity distribution and/or the attenuation map. In some embodiments, the methods disclosed herein include using the activity distribution and/or the attenuation map to train one or more GANs to produce additional activity distributions and/or additional attenuation maps. In some embodiments, the methods disclosed herein include segmenting one or more of the plurality of real MR images and/or the plurality of real CT images to produce at least one set of segmented MR images and/or at least one set of segmented CT images. In some embodiments, the methods disclosed herein include producing the set of segmented MR images and/or the set of segmented CT images using at least one convolutional neural network (CNN), at least one recurrent neural network (RNN), at least one feedforward neural network, at least one residual neural network, and/or at least one autocoder network. In some embodiments, the methods disclosed herein include validating the set of segmented MR images and/or the set of segmented CT images using one or more separately segmented MR images and/or separately segmented CT images as ground truth images.

In some embodiments, the methods disclosed herein include identifying one or more regions-of-interest in images in the set of segmented MR images and/or the set of segmented CT images to produce at least one set of regions-of-interest in the images. In certain embodiments, the methods disclosed herein include filling at least some of the regions-of-interest in the set of regions-of-interest in the images with at least one selected activity concentration to produce at least one set of filled images. In some embodiments, the methods disclosed herein include computing the selected activity concentration from one or more real single photon emission computed tomography (SPECT) and/or one or more real positron emission tomography (PET) images. In some embodiments, the methods disclosed herein include filling one or more regions of images in the set of segmented MR images and/or the set of segmented CT images with the selected activity concentration to produce at least one attenuation map. In certain embodiments, the methods disclosed herein include generating one or more regions-of-interest maps for quantification from the set of segmented MR images and/or the set of segmented CT images. In some embodiments, the methods disclosed herein include quantifying the activity distribution from one or more real single photon emission computed tomography (SPECT) and/or one or more real positron emission tomography (PET) images. In certain embodiments, the methods disclosed herein include inputting at least one noise vector into a generator network of the GAN to produce the plurality of synthetic MR images and/or the plurality of synthetic CT images. In some embodiments, the methods disclosed herein include training a discriminator network of the GAN to distinguish between the real MR images and the synthetic MR images and/or between the real CT images and the synthetic CT images.

In certain embodiments, the methods disclosed herein include initializing one or more learnable parameters in the generator and/or the discriminator network of the GAN. In some embodiments, the learnable parameters in the generator network comprise trainable learnable parameters. In some embodiments, the learnable parameters in the discriminator network comprise frozen learnable parameters.

In some embodiments, the GAN produces a plurality of synthetic two dimensional (2D) MR images and/or a plurality of synthetic 2D CT images. In certain embodiments, the GAN produces a plurality of synthetic three dimensional (3D) MR images and/or a plurality of synthetic 3D CT images. In some embodiments, the GAN comprises at least one feedforward generator network. In some embodiments, the plurality of real MR images and/or the plurality of real CT images comprise real brain images. In certain embodiments, the real brain images comprise one or more real control brain images, one or more real Parkinson's Disease (PD) images, and/or one or more real scans without evidence of a dopaminergic deficit (SWEDD) images.

In certain embodiments, a generator network and/or a discriminator network of the GAN comprises a two dimensional (2D) convolutional neural network. In some embodiments, a generator network and/or a discriminator network of the GAN comprises a three dimensional (3D) convolutional neural network. In certain embodiments, the methods disclosed herein include determining at least one cycle consistency loss. In certain embodiments, the cycle consistency loss comprises at least one forward cycle consistency loss and/or at least one backward cycle consistency loss. In certain embodiments, the real MR images, the synthetic MR images, the real CT images, and/or the synthetic CT images are two dimensional (2D) images. In certain embodiments, the real MR images, the synthetic MR images, the real CT images, and/or the synthetic CT images are three dimensional (3D) images.

In some aspects, the present disclosure provides a system, comprising at least one controller that comprises, or is capable of accessing, computer readable media comprising non-transitory computer executable instructions which, when executed by at least one electronic processor, perform at least: training at least one generative adversarial network (GAN) with data from a plurality of real and/or synthetic magnetic resonance (MR) images and/or data from a plurality of real and/or synthetic computed tomography (CT) images to produce at least one activity distribution and/or at least one attenuation map.

In some aspects, the present disclosure provides a system, comprising at least one controller that comprises, or is capable of accessing, computer readable media comprising non-transitory computer executable instructions which, when executed by at least one electronic processor, perform at least: training at least one generative adversarial network (GAN) with data from a plurality of synthetic magnetic resonance (MR) images and/or data from a plurality of synthetic computed tomography (CT) images to produce at least one activity distribution and/or at least one attenuation map; and, using the activity distribution and/or the attenuation map to conduct at least one medical imaging simulation.

In certain aspects, the present disclosure provides a system, comprising at least one controller that comprises, or is capable of accessing, computer readable media comprising non-transitory computer executable instructions which, when executed by at least one electronic processor, perform at least: training at least one generative adversarial network (GAN) with non-mean data from a plurality of real magnetic resonance (MR) images and/or non-mean data from a plurality of real computed tomography (CT) images to produce a plurality of synthetic MR images and/or a plurality of synthetic CT images.

In other aspects, the present disclosure provides a system, comprising at least one controller that comprises, or is capable of accessing, computer readable media comprising non-transitory computer executable instructions which, when executed by at least one electronic processor, perform at least: training at least one generative adversarial network (GAN) with data from a plurality of real magnetic resonance (MR) images and/or data from a plurality of real computed tomography (CT) images to produce a plurality of synthetic MR images and/or a plurality of synthetic CT images that comprises substantially accurate anatomical information; and, converting the plurality of synthetic MR images and/or the plurality of synthetic CT images to at least one activity distribution and/or at least one attenuation map.

In some aspects, the present disclosure provides a computer readable media comprising non-transitory computer executable instruction which, when executed by at least electronic processor perform at least: training at least one generative adversarial network (GAN) with data from a plurality of real and/or synthetic magnetic resonance (MR) images and/or data from a plurality of real and/or synthetic computed tomography (CT) images to produce at least one activity distribution and/or at least one attenuation map.

In certain aspects, the present disclosure provides a computer readable media comprising non-transitory computer executable instruction which, when executed by at least electronic processor perform at least: training at least one generative adversarial network (GAN) with data from a plurality of synthetic magnetic resonance (MR) images and/or data from a plurality of synthetic computed tomography (CT) images to produce at least one activity distribution and/or at least one attenuation map; and, using the activity distribution and/or the attenuation map to conduct at least one medical imaging simulation.

In some aspects, the present disclosure provides a computer readable media comprising non-transitory computer executable instruction which, when executed by at least electronic processor perform at least: training at least one generative adversarial network (GAN) with non-mean data from a plurality of real magnetic resonance (MR) images and/or non-mean data from a plurality of real computed tomography (CT) images to produce a plurality of synthetic MR images and/or a plurality of synthetic CT images.

In certain aspects, the present disclosure provides a computer readable media comprising non-transitory computer executable instruction which, when executed by at least electronic processor perform at least: training at least one generative adversarial network (GAN) with data from a plurality of real magnetic resonance (MR) images and/or data from a plurality of real computed tomography (CT) images to produce a plurality of synthetic MR images and/or a plurality of synthetic CT images that comprises substantially accurate anatomical information; and, converting the plurality of synthetic MR images and/or the plurality of synthetic CT images to at least one activity distribution and/or at least one attenuation map.

In some embodiments of the system or computer readable media disclosed herein, the instructions further perform at least: using the activity distribution and/or the attenuation map to conduct one or more medical imaging simulations. In some embodiments of the system or computer readable media disclosed herein, the imaging simulations comprise a known ground truth. In certain embodiments of the system or computer readable media disclosed herein, the instructions further perform at least: using the medical imaging simulations to perform at least one image-based task. In some embodiments of the system or computer readable media disclosed herein, the medical imaging simulations comprise single photon emission computed tomography (SPECT) and/or positron emission tomography (PET). In some embodiments of the system or computer readable media disclosed herein, images in the plurality of real MR images and/or the plurality of real CT images are of at least one anatomical part of a plurality of reference subjects. In some embodiments of the system or computer readable media disclosed herein, the plurality of synthetic MR images and/or the plurality of synthetic CT images comprises a substantially normal distribution of anatomical variability present in the plurality of reference subjects. In some embodiments of the system or computer readable media disclosed herein, the plurality of reference subjects comprises normal and/or abnormal subjects. In some embodiments of the system or computer readable media disclosed herein, the plurality of synthetic MR images and/or the plurality of synthetic CT images comprises substantially accurate anatomical information. In some embodiments of the system or computer readable media disclosed herein, the data from the plurality of synthetic MR images and/or the data from the plurality of synthetic CT images comprises non-mean data.

In some embodiments of the system or computer readable media disclosed herein, the instructions further perform at least: using the substantially accurate anatomical information to produce at least one regions-of-interest map. In some embodiments of the system or computer readable media disclosed herein, the anatomical part is selected from the group consisting of: an organ, a torso, an abdomen, an extremity, and a tumor. In some embodiments of the system or computer readable media disclosed herein, the activity distribution and/or the attenuation map models at least one normal population and/or at least one diseased population. In some embodiments of the system or computer readable media disclosed herein, the organ comprises a brain.

In some embodiments of the system or computer readable media disclosed herein, the instructions further perform at least: using at least one compression technique to compress dimensionality of one or more of the plurality of real and/or synthetic MR images and/or the plurality of real and/or synthetic CT images. In some embodiments of the system or computer readable media disclosed herein, the compression technique comprises using at least one autoencoder network. In some embodiments of the system or computer readable media disclosed herein, the autoencoder network comprises at least one encoder that maps input MR images and/or input CT images to at least one feature map that comprises a lower dimensional space than the input MR images and/or the input CT images to produce compressed images. In some embodiments of the system or computer readable media disclosed herein, the autoencoder network comprises at least one decoder that maps the compressed images to the input MR images and/or to the input CT images. In some embodiments of the system or computer readable media disclosed herein, the instructions further perform at least: training the autoencoder network using an unsupervised training technique. In some embodiments of the system or computer readable media disclosed herein, the compression technique does not use down-sampling. In some embodiments of the system or computer readable media disclosed herein, the autoencoder network comprises a stacked configuration comprising multiple autoencoders. In some embodiments of the system or computer readable media disclosed herein, the autoencoder network comprises using one or more hyperparameters.

In some embodiments, the system or computer readable media disclosed herein include using at least one training procedure that comprises a mean square error (MSE) as a cost function and/or a sparsity regularizer. In some embodiments of the system or computer readable media disclosed herein, the autoencoder network comprises a multi-layer convolutional neural network (CNN) configuration that compresses and abstracts a given image being processed two or more times. In some embodiments of the system or computer readable media disclosed herein, the instructions further perform at least: using at least one convolutional neural network (CNN) and/or at least one cycle consistent GAN (CycleGAN) to produce the activity distribution and/or the attenuation map. In some embodiments of the system or computer readable media disclosed herein, the instructions further perform at least: validating anatomical realism and/or accuracy of one or more of the plurality of synthetic MR images, the plurality of synthetic CT images, the activity distribution, and/or the attenuation map. In some embodiments of the system or computer readable media disclosed herein, the instructions further perform at least: quantifying one or more activity distributions in one or more single photon emission computed tomography (SPECT) and/or positron emission tomography (PET) images to convert the plurality of synthetic MR images and/or the plurality of synthetic CT images to the activity distribution and/or the attenuation map. In some embodiments of the system or computer readable media disclosed herein, the instructions further perform at least: using the activity distribution and/or the attenuation map to train one or more GANs to produce additional activity distributions and/or additional attenuation maps.

In some embodiments of the system or computer readable media disclosed herein, the instructions further perform at least: segmenting one or more of the plurality of real MR images and/or the plurality of real CT images to produce at least one set of segmented MR images and/or at least one set of segmented CT images. In some embodiments of the system or computer readable media disclosed herein, the instructions further perform at least: producing the set of segmented MR images and/or the set of segmented CT images using at least one convolutional neural network (CNN), at least one recurrent neural network (RNN), at least one feedforward neural network, at least one residual neural network, and/or at least one autocoder network. In some embodiments of the system or computer readable media disclosed herein, the instructions further perform at least: validating the set of segmented MR images and/or the set of segmented CT images using one or more separately segmented MR images and/or separately segmented CT images as ground truth images. In some embodiments of the system or computer readable media disclosed herein, the instructions further perform at least: identifying one or more regions-of-interest in images in the set of segmented MR images and/or the set of segmented CT images to produce at least one set of regions-of-interest in the images. In some embodiments of the system or computer readable media disclosed herein, the instructions further perform at least: filling at least some of the regions-of-interest in the set of regions-of-interest in the images with at least one selected activity concentration to produce at least one set of filled images. In some embodiments of the system or computer readable media disclosed herein, the instructions further perform at least: computing the selected activity concentration from one or more real single photon emission computed tomography (SPECT) and/or one or more real positron emission tomography (PET) images.

In some embodiments of the system or computer readable media disclosed herein, the instructions further perform at least: filling one or more regions of images in the set of segmented MR images and/or the set of segmented CT images with the selected activity concentration to produce at least one attenuation map. In some embodiments of the system or computer readable media disclosed herein, the instructions further perform at least: generating one or more regions-of-interest maps for quantification from the set of segmented MR images and/or the set of segmented CT images. In some embodiments of the system or computer readable media disclosed herein, the instructions further perform at least: quantifying the activity distribution from one or more real single photon emission computed tomography (SPECT) and/or one or more real positron emission tomography (PET) images. In some embodiments of the system or computer readable media disclosed herein, the instructions further perform at least: inputting at least one noise vector into a generator network of the GAN to produce the plurality of synthetic MR images and/or the plurality of synthetic CT images.

In some embodiments of the system or computer readable media disclosed herein, the instructions further perform at least: training a discriminator network of the GAN to distinguish between the real MR images and the synthetic MR images and/or between the real CT images and the synthetic CT images. In some embodiments of the system or computer readable media disclosed herein, the instructions further perform at least: initializing one or more learnable parameters in the generator and/or the discriminator network of the GAN. In some embodiments of the system or computer readable media disclosed herein, the learnable parameters in the generator network comprise trainable learnable parameters. In some embodiments of the system or computer readable media disclosed herein, the learnable parameters in the discriminator network comprise frozen learnable parameters. In some embodiments of the system or computer readable media disclosed herein, the GAN produces a plurality of synthetic two dimensional (2D) MR images and/or a plurality of synthetic 2D CT images. In some embodiments of the system or computer readable media disclosed herein, the GAN produces a plurality of synthetic three dimensional (3D) MR images and/or a plurality of synthetic 3D CT images.

In some embodiments of the system or computer readable media disclosed herein, the GAN comprises at least one feedforward generator network. In some embodiments of the system or computer readable media disclosed herein, the plurality of real MR images and/or the plurality of real CT images comprise real brain images. In some embodiments of the system or computer readable media disclosed herein, the real brain images comprise one or more real control brain images, one or more real Parkinson's Disease (PD) images, and/or one or more real scans without evidence of a dopaminergic deficit (SWEDD) images.

In some embodiments of the system or computer readable media disclosed herein, a generator network and/or a discriminator network of the GAN comprises a two dimensional (2D) convolutional neural network. In some embodiments of the system or computer readable media disclosed herein, a generator network and/or a discriminator network of the GAN comprises a three dimensional (3D) convolutional neural network. In some embodiments of the system or computer readable media disclosed herein, the instructions further perform at least: determining at least one cycle consistency loss. In some embodiments of the system or computer readable media disclosed herein, wherein the cycle consistency loss comprises at least one forward cycle consistency loss and/or at least one backward cycle consistency loss. In some embodiments of the system or computer readable media disclosed herein, the synthetic MR images, the real CT images, and/or the synthetic CT images are two dimensional (2D) images. In some embodiments of the system or computer readable media disclosed herein, the real MR images, the synthetic MR images, the real CT images, and/or the synthetic CT images are three dimensional (3D) images.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 shows example activity distributions (right) and attenuation maps (middle) generated from one patient's MRI images (left) according to one embodiment.

FIG. 2 shows 2D trans-axial cross-sections of 3D segmented brain regions of the caudate and putamen according to one embodiment. In particular, the original image is shown in (panel a). The delineations by both an embodiment of a method disclosed herein and FreeSurfer are compared in (panel b). The segmentations by this method are shown in (panel c). The segmentations by FreeSurfer, which was used as ground truth, are shown in (panel d).

FIG. 3 is a schematic illustration of a 3D U-net architecture according to one embodiment.

FIG. 4 is a schematic illustration of a generative adversarial network (GAN) system architecture according to one embodiment.

FIG. 5 schematically shows the structure of an autoencoder according to an exemplary embodiment. As shown, the process of M→N is a compressing conversion (M>N). The later process N→M is a decompressing conversion.

FIG. 6 shows an example of a 128×128 image that is compressed to a feature vector of 256 elements by an encoder and then recovered after decoder according to one embodiment.

FIG. 7 schematically shows an exemplary architecture of an autoencoder involving CNN layers according to one embodiment.

FIG. 8 schematically shows an illustration of a CycleGAN network architecture according to one embodiment.

FIG. 9 schematically shows exemplary steps of computing cycle consistency loss according to one embodiment.

FIG. 10 schematically shows example images from a CycleGAN according to one embodiment. An original MRI image, the attenuation map generated from MRI using G1, and the MRI image generated using G2 from the G1 generated attenuation map is shown in (panel a). An activity distribution, the synthetic MRI generated from the activity distribution using G2, and the activity distribution generated using G1 from the G2 generated MRI is shown in (panel b).

FIG. 11 schematically depicts steps using a GAN to directly generate an activity distribution and attenuation map without using an MRI image according to one embodiment.

FIG. 12 shows randomly generated synthetic brain MR images in panels a, b, d, and e. These synthetic examples are compared to examples of real brain MR images in panels c and f.

FIG. 13 are image intensity histograms of real (panel a) and synthetic (panel b) MR images.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, computer readable media, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Non-Mean: As used herein, “non-mean” in the context of data refers to data that is not based on the arithmetic mean or average of one or more characteristics, attributes, or parameters of a given sample.

Pathology: As used herein, “pathology” refers to a deviation from a normal state of health, such as a disease (e.g., neoplastic or non-neoplastic diseases), abnormal condition, or disorder.

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., known ocular or other pathology and/or the like).

### DETAILED DESCRIPTION

Simulation is an important tool for evaluating SPECT and PET reconstruction and compensation methods. In a simulation study, the ground truth is known, and by controlling which factors are included in the simulation, one can quantify the effects of different factors such as attenuation and scatter. Since quantitative estimation tasks are performed in populations of patients who exhibit a wide range of variability in terms of anatomy and tracer distribution, and since the accuracy and precision of the quantification depend on these same factors, quantitative SPECT and PET methods should be evaluated and optimized using a phantom population with a range of anatomies and distributions. It is typically not possible to use physical phantoms in population studies because physical phantoms generally only provide a limited number of rigid organ compartments and do not model the variation of patient populations. Additionally, the plastic shell around the compartments are not present in human data. In contrast, digital phantoms allow for the modeling of anatomical variations and do not suffer from such constraints. However, the total number of phantoms is still limited. Thus, the use of existing digital brain phantoms is not enough to overcome the overfitting issue that often occurs in the training of neural networks. Accordingly, in some aspects of the present disclosure, a generative adversarial network (GAN) is used to produce a large number (i.e. as many as needed) of synthetic structural brain MRI images that can, for example, be readily converted to brain phantoms for SPECT and PET simulation.

In overview, the present disclosure provides methods to directly convert existing patient MRI images to activity distributions and attenuation maps in certain embodiments. In some aspects, these methods can be used for converting synthetic MRI images to activity distributions and attenuation maps that can be used in simulation studies. Their output can also be used to train a CycleGAN, as described herein, for same purpose. In some embodiments, GANs are trained to produce synthetic structural MRI images, which are validated to evaluate their realism and accuracy. Both analytical methods and human observer studies are optionally used for such validation. In certain embodiments, synthetic MRI images are converted into the activity distributions and attenuation maps needed for SPECT and PET simulation using the methods disclosed herein. The phantom population generated using the methods disclosed herein can also be used to train a GAN to directly generate activity distributions and attenuation maps in some embodiments. To validate a given phantom population, selected phantoms can be used in Monte Carlo simulations to generate SPECT and PET data is some applications.

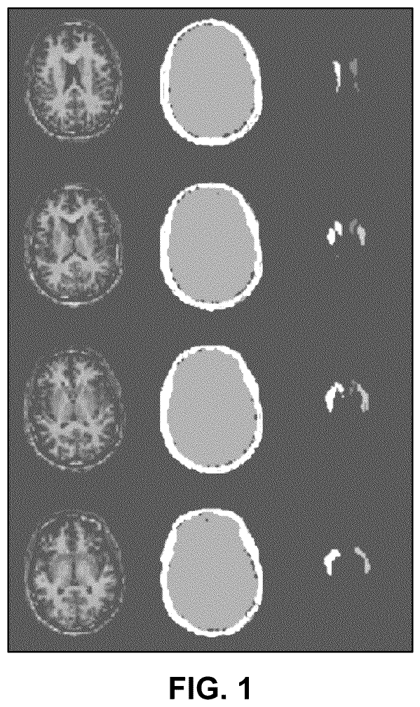

In some aspects, the present disclosure provides methods to produce activity distributions and attenuation maps from real patient MRI images. In some embodiments, these methods include segmenting the real MRI images to produce the activity distributions and attenuation maps. In certain of these embodiments, for example, the MRI images are segmented and then each region-of-interest is filled with a desired activity concentration. The activity concentration is optionally computed from PET or SPECT images. In certain of these embodiments, attenuation maps are generated by filling each region of segmented MRI images with the corresponding attenuation coefficient. The regions-of-interest maps for quantification can also be generated from segmented MRI images FreeSurfer or another imaging software package. To illustrate, FIG. 1 shows one example of activity distribution and attenuation map generated from one patient's MRI images. The activity distribution mimic those seen in a DATscan SPECT imaging. To generate skull in the attenuation map, the brain in the MRI images was first removed using a mask from the FreeSurfer skull-stripping. Then, a thresholding method was applied to the rest of the image to generate the skull volume-of-interest (VOI), which was then filled with corresponding attenuation coefficient of the bone.

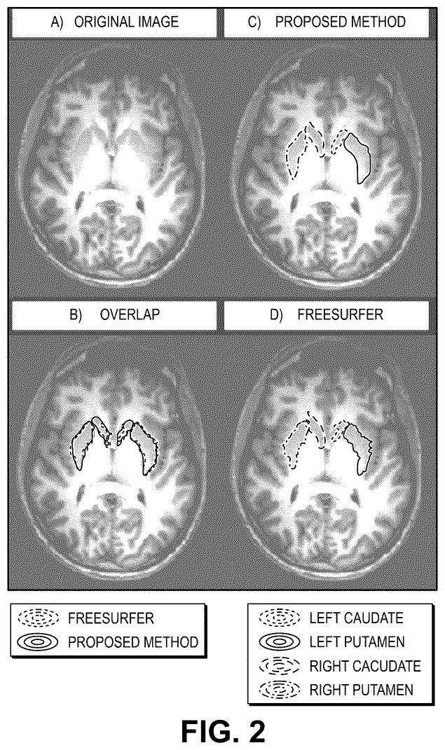

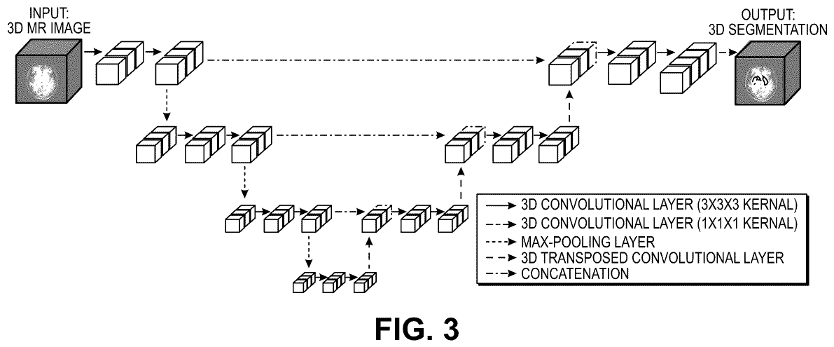

Software packages such as FreeSurfer, SPM, FSL, and MRIClouId can take hours or days to segment one full 3D brain MRI image. They are also sensitive to noise, artifacts, and contrast variation. Therefore, it can be very time consuming to segment MRI images. To address this, a deep learning approach for fully automated three-dimensional (3D) segmentation of striatal structures on, for example, brain MRI images can be used. In some of these embodiments, the methods use a deep 3D U-net architecture (FIG. 3) and are trained on training and validation sets of MRI images from real patients and then evaluated on test sets of MRI images from patients. The network can be trained by minimizing a class-weighted cross-entropy loss function with, for example, Adam, a first-order stochastic gradient-based optimization algorithm. To illustrate, FIG. 2 shows 2D trans-axial cross-sections of the 3D segmented brain regions of the caudate and putamen. Overall, certain embodiments of the methods disclosed herein delineate brain regions in high agreement with the ground truth from segmentation based on FreeSurfer (FIG. 2c). The deep learning segmentation is also very fast, taking less than a second (e.g., 0.77±0.04 seconds) per brain MRI image in certain embodiments.

In certain embodiments, the artificial neural network is further trained to segment other anatomical regions (e.g., brain regions from MRI images using FreeSurfer results as ground truth). In these embodiments, the regions can include white matter, brain stem, thalamus, hippocampus, cerebellum cortex, cerebellum white matter, frontal cortex, temporal cortex, occipital cortex, parietal cortex, cingulate cortex, and the like. In these embodiments, MRI images can be randomly partitioned into training, validation and testing sets using a training/validation/test split of 60%/20%/20%. Optionally, the network can be trained by minimizing a class-weighted cross-entropy loss function with, for example, Adam.

In some embodiments, the deep learning-based segmentation methods are trained on real MRI images and activity distributions in, for example, brain regions are quantified from SPECT and PET images. Then each region in the segmented MRI images are filled with corresponding activity. The attenuation maps are also generated based on the skull VOIs obtained from the trained network in some of these embodiments.

In some of these embodiments, the methods disclosed herein include training convolutional neural networks to produce activity distributions and attenuation maps. In certain of these embodiments, the data is randomly partitioned into training, validation and testing sets using a training/validation/test split of, for example, 60%/20%/20%. Optionally, a 3D U-Net deep convolutional neural network with, for example, architecture shown in FIG. 3 is trained on the direct image-to-image translation task of mapping the input MRI image to the corresponding activity distribution. A separate CNN is also optionally trained with MRI images as input and attenuation maps as the output. Training a CNN to produce both activity distribution and attenuation map simultaneously from the MRI image is also optionally utilized. The trained CNN can be used to convert synthetic MRI images to SPECT or PET activity distributions and attenuation maps.

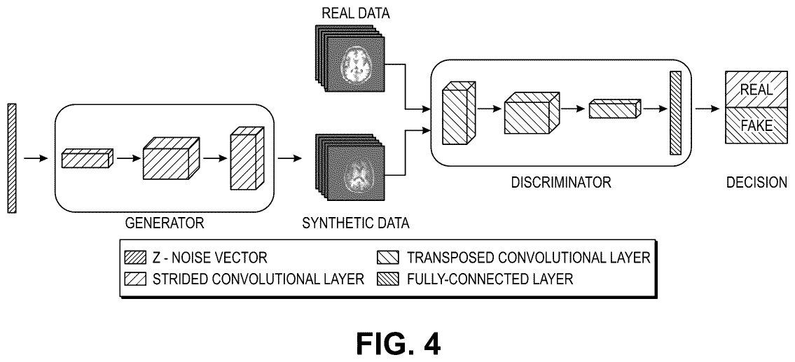

In certain embodiments, GANs are trained to produce synthetic brain structural MRI images. For example, this technique is optionally used to produce synthetic structural brain MRI images, which can be used in subsequent procedures to generate activity distributions and attenuation maps. The GAN typically consists of two neural networks: a generator and a discriminator network. The generator is given a noise vector (e.g. Gaussian noise) as input and generates synthetic images (e.g., brain MRI images) as an output. The discriminator network is trained to distinguish the synthetic examples created by the generator from real brain MRI images. The generator is then typically trained to create synthetic brain MRI images that are realistic enough to fool the discriminator. The two networks are typically trained in this adversarial manner to generate realistic synthetic trans-axial cross-sections of brain MRI images. An exemplary GAN is shown in FIG. 4. The network architecture is composed of strided convolutional layers and transposed convolutional layers that perform down-sampling and up-sampling in the generator and discriminator networks, respectively. Batch normalization is used throughout the generator and discriminator networks to stabilize the adversarial learning process. Dropout with a drop probability of 0.5 is typically used in the last fully-connected layer of the discriminator before the classification of real and synthetic examples. In some embodiments, the GAN is trained to produce MRI images of control, PD, and SWEDD separately using corresponding real MRI images. To illustrate, some basic training steps optionally are:
1) Initialize the learnable parameters in the generator and discriminator network and use the generator to produce fake MRI images. At this point, the fake images are typically easily distinguished from real images.
2) Train the discriminator to discern real MRI images from fake images produced by the generator. This is generally a classification problem where there are two categories: real and fake images. In some embodiments, it can use the true class labels representing real and fake to train the discriminator on classifying real and fake images, respectively.
3) After updating the learnable parameters of the discriminator network for one training step, the learnable parameters of the generator network are then typically trained and updated for the next training step while freezing learnable parameters in the discriminator network. While the goal of the discriminator network is typically to correctly classify fake from real MRI images, the goal of the generator network is generally to fool the discriminator into thinking that the fake generated MRI images are real images. When training the generator, the class labels of the fake images are flipped to belong to the real class label in some embodiments. Typically, the generator (which has trainable learnable parameters) and the discriminator (which has frozen learnable parameters) networks are chained together and trained on the classification task where the generator's goal is to produce realistic images that are classified as being real images by the discriminator.
4) The discriminator and generator networks are typically trained together in this adversarial manner until the generator can produce very visually realistic MRI images.

FIG. 12 shows example MRI images generated using the GAN as compared with real MRI images. The GAN can generate any 2D MRI image slice of, for example, a human head, and the results resemble real MRI images. The GAN-based approach can be extended to generate 3D images.

In some embodiments, a feedforward fully-connected GAN is trained to produce synthetic brain MRI images. In one of these exemplary embodiments, the generator network is a traditional feedforward fully-connected network that converts the input noise vector, z∈ [symbol] ^{N}; to the output that has a dimensionality of M²×1. The output is then reshaped to an M×M pixel image. The feedforward generator network may have multiple fully-connected layers and is followed by a discriminator network to compose a GAN. In some of these embodiments, the training of the GAN consists of two alternating steps:
1) The discriminator network is trained given real MRI brain images with labels (e.g., 1 for the real image, 0 for the synthetic image). Such training is a supervised classification problem in some embodiments.
2) The generator network is then followed by the discriminator network trained in the previous step to update the parameters in the generator network only (e.g., fix the parameters in the discriminator network) during the training, meanwhile all output labels are set to 1, because the images produced by the generator network are intended to be like real images as much as possible.
3) Using the generator network obtained in step 2) to generate artificial, for example, brain MRI images, and then combine those with real images to repeat step 1) in order to train the discriminator network for the second round. The discriminator and generator networks are then trained alternately until the generative net can produce visually realistic images.

Some embodiments involve training a 2D convolutional GAN. In certain of these applications, the generator network is a 2D convolutional network to convert any N×N random-number array to a M×M pixel image. The generator network may contain multiple convolutional layers with each layer having a different size and number of filter kernels. In some of these embodiments, the discriminator network is also a 2D convolutional neural network whose input is 2D images. Hence, there is no reshaping from 1D to 2D in these embodiments. The training steps of such a 2D convolutional GAN are typically the same as others described herein. Typically, the optimal dimensionality of the input noise vector, z∈ [symbol] ^{N}, is selected for a given GAN application. Using a very small value for N (low dimensional noise z vector) may restrict the ability of the GAN to express the full diversity present in the data and lead to generated images that are very similar to each other. Contrarily, using a very large value for N (high dimensional noise z vector) may add difficulty to the training process and slow down the training convergence.

Some embodiments involve training GANs to directly produce 3D synthetic structural MRI images. Certain of these embodiments involve the use of autoencoders to compress 3D images to increase training efficiency. In some of these embodiments, for example, phantoms are generated in 3D for SPECT and PET simulation. For example, a GAN can generate synthetic MRI images for any 2D slice of the brain. Optionally, the GANs are extended to directly generate synthetic 3D MRI images. The generator and discriminator networks are extended from 2D to 3D convolutional neural networks. In these embodiments, the input training data is 3D images, and the discriminator discriminates real from fake 3D images as opposed to 2D images. However, in these embodiments, the dimensionality of the 3D MRI image volumes of the brain are large (e.g., 256×256×256) and 3D convolutional layers are more computationally expensive than 2D convolution layers. In some instances, the hardware limitations in memory and GPU speed can be prohibitive. To address these limitations, a method to reduce the computational complexity of the training is implemented by compressing the 3D MRI image volumes to a lower dimensional feature space. In some of these embodiments, this is achieved by using an autoencoder network that consists of both an encoder and a decoder.

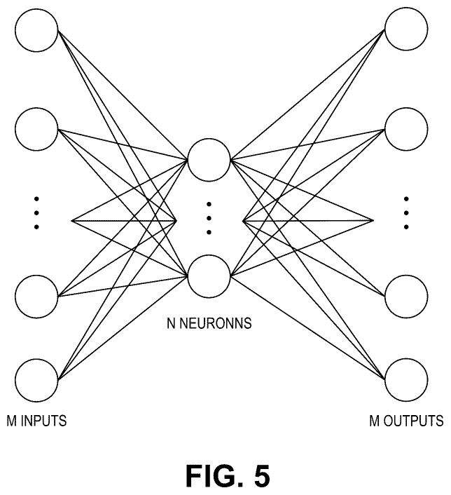

To illustrate, the encoder maps the 3D input image to a feature map of a lower dimensional space (i.e. the compressed image) in certain of these embodiments. The decoder maps the compressed image back to the original 3D input image. The GAN is then trained to create the lower dimensional feature maps instead of the full 3D images. Since the feature maps have a reduced number of voxels, the GAN can converge more quickly with less computational load than when generating full 3D image volumes. Finally, the feature maps generated from the GAN can be applied to a decoder to generate 3D MRI images. In some embodiments, the autoencoder consists of an encoder and a decoder, as illustrated in FIG. 5, which shows a three-layer fully-connected neural network. As shown, the first layer compresses the input image with M voxels to a vector with fewer elements (N variables). Since M>N, the N outputs of the first layer can be thought of as the feature map of the original image in a lower dimensional space. The second layer recovers the original 3D image from the lower dimensional feature map. The autoencoder network is typically trained in an unsupervised fashion where the input image is mapped back to itself in the output. As such, there is generally no need for labeled data when training the autoencoder. In some of these embodiments, the autoencoder network does not perform a simple down-sampling on the input image. In certain embodiments, the encoder of the autoencoder learns to map the original 3D images to an arbitrary lower dimensional space from which the decoder can map back to the original 3D image space. This allows the images to be compressed to a feature space with a much lower dimensionality (i.e. very few variables (N<<M)) than what simple image down-sampling can achieve.

In some embodiments, the compression efficiency of an autoencoder is further improved by using multiple layers. To reduce training difficulty, one layer is typically trained at a time instead of training a multi-layer neural network directly. For example, in first step, a three-layer neural network having the same structure as FIG. 5 is trained to compress 3D images to a lower dimensional feature map space. Next, these feature maps can be used as the inputs and outputs to train another autoencoder that will further compress those feature maps. By using stacked autoencoders, high-dimensional data compressed to low-dimensional data becomes less challenging.

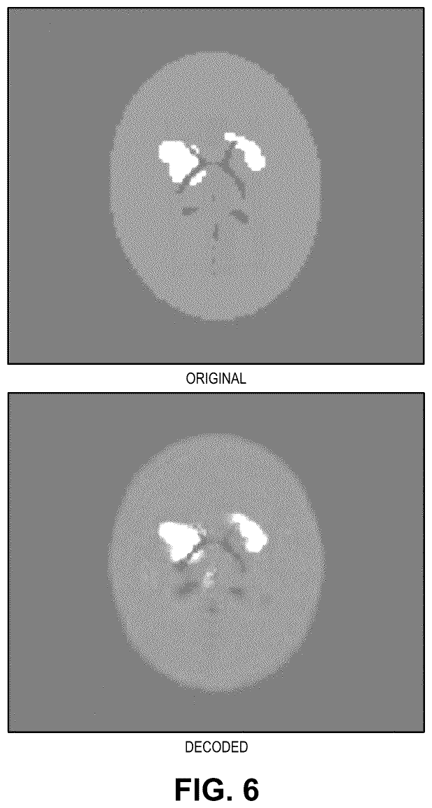

FIG. 6 shows an example of a 128×128 image that is compressed to a feature vector of 256 elements by an encoder and then recovered after decoder. One can see there is certain information loss in the decoded image. To improve this, mean squared error (MSE) can be used as the cost function. In addition, since there are often fewer voxels in the image representing the useful information than the voxels representing the background, it is useful to add a sparsity regularizer to enforce a constraint on the sparsity of the output layer in some of these embodiments. A common sparsity regularizer is the Kullback-Leibler divergence, as follows:

$${Ω}_{sparsity} = {\sum }_{i = 1}^{D ( 1 )} KL ( ρ ‖ ) = {\sum }_{i = 1}^{D ( 1 )} [ ρ log ( ) + ( 1 - ρ ) log ( ) ] ,$$

where [symbol] and ρ are the average activation value of a neuron i and its desired value, respectively. If also considering adding an L2 regularization Ω_{weights} term to avoid overfitting, the final cost function becomes:

Cost=MSE+λ·Ω_{weights}+β·Ω_{sparsity},

where λ is the coefficient for L2 regularization and β is the coefficient for sparsity regularization. There is typically no general rule for selecting value for λ and β, so systematic testing is used to find the most appropriate value for these hyperparameters is some embodiments.

Alternatively, instead of using only fully connected layers, an encoder that involves multiple convolutional layers is optionally implemented. To illustrate, in a multi-layer CNN, the characteristics in the original image are extracted by the first CNN layer in terms of the feature maps, and these feature maps are further abstracted by the next CNN layers and finally summarized to very limited number of characteristics in certain embodiments. In other words, the operation of the CNN layers is a continuous process of compressing and abstracting in these embodiments. Therefore, it can be utilized to perform image compressing as an image encoder.

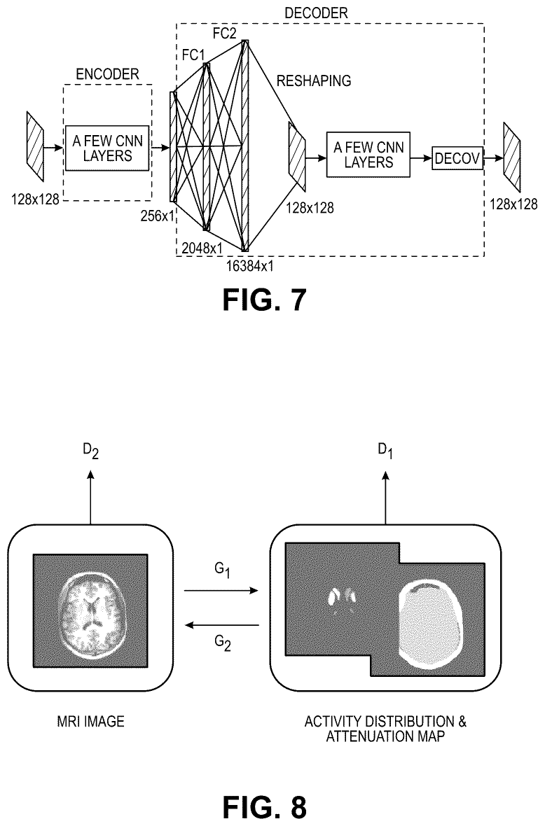

As a further illustration, the overall network structure of a CNN autoencoder is schematically depicted in FIG. 7 for a 2D example. As shown, the first two fully-connected layers in the autoencoder shown in FIG. 7 are replaced by a few CNN layers to form an encoder which compresses, for example, a 128×128 image to a 256×1 vector. The following two fully-connected layers up-sample the 256 variables back to 16,384 variables which is equivalent to the counterpart in the autoencoder in FIG. 5. Typically, transposed convolutions are not used to perform the up-sampling, because it adds many zeros to the compressed data, which is not efficient. The output of the second fully-connected layer is reshaped to a 128×128 array, and then passes through an image optimizer consisting of a few CNN layers (usually 2 CNN layers can do denoising and resolution enhancement fairly well). Finally, a recovered image is obtained by performing a deconvolution in this exemplary embodiment.

Certain embodiments include validating the accuracy of synthetic structural MRI images produced using the methods disclosed herein. In some of these embodiments, for example, the anatomical realism and accuracy of the synthetic MRI images is validated using existing brain MRI segmentation and analysis software, such as FreeSurfer and SPM. To illustrate, the discriminator in the GAN described herein is optionally trained to distinguish the synthetic examples created by the generator from real brain MRI images. However, it is typically useful to validate the realism of the synthetic MRI images and the anatomical accuracy of the brain structures in the images using independent methods. Since new synthetic MRI images are generated, their accuracy cannot generally be validated using common methods such as mean square errors or structural similarity index (SSIM) as there is no corresponding ground truth for the synthetic images. There are existing MRI segmentation and analysis software such as FreeSurfer, SPM, and MRICloud, among others, that can not only segment MRI images, but also provide statistical analysis of structural abnormalities in the brain. Accordingly, in some embodiments, these software are used by running both the real MRI images and the synthetic MRI images through the analysis. The structural volumes and other outputs from these programs can then be statistically compared with one another. The abnormalities reported by these programs, such as atrophy in a normal brain, can also be used as an indication of any inaccuracy in the synthetic MRI images in some of these embodiments.

Statistical analysis can be performed using software, such as SPSS (version 25, IBM). The FreeSurfer and SPM output on real human MRI images can be used as the gold standard and compared with the results from synthetic MRI images. For example, the compared volumes of various brain structures, include white matter, brain stem, thalamus, hippocampus, cerebellum cortex, cerebellum white matter, frontal cortex, temporal cortex, occipital cortex, parietal cortex, and cingulate cortex, among other structures. The thickness of various cerebral cortex regions can also be measured and compared. Descriptive dot-plots of these quantitative measures can also be created to visually examine the outcomes. In some embodiments, the comparison is performed using multivariate analysis of variance (MANOVA). Then, separate analysis of variance (ANOVA) are carried out for each brain region. Linear regression is then typically performed between synthetic data and real human data. Data transformations is optionally performed when dependent data are not normally distributed.

In certain embodiments, the synthetic MRI images are evaluated using human observers. In some of these embodiments, for example, a high quality NEC color-calibrated (using AAPM standards) LCD monitor is used with carefully controlled brightness in a dark room. In certain embodiments, ROC display software is used to perform the studies. In some of these evaluations, images are separated into a unit containing two blocks: “training” and “testing”. Each unit contains images from real or synthetic images with randomized orders. The purpose of the “training” block is to reset and to stabilize the observer's internal decision criteria. Generally, it is desirable to have a large number of training image sets for better stabilization. However, observer fatigue and practical time constraints on the length of an observer study can limit the maximum number of images evaluated. Therefore, some embodiments use 20 images for training and 40 images for testing in each unit. In these embodiments, each study may contain two units and each observer may spend 10 to 20 seconds on each image. The study length is typically to about 60 minutes per observer to allow for stable observation performance without fatigue. Typically, blind studies are performed, during which a given observer is asked to score the images on a 5-point scale as 5=real image, 4=probably real, 3=not sure, 2=probably synthetic, and 1=definitely synthetic.

The goal of these studies is to evaluate if the human observers can separate the synthetic images from real images. Ideally, the histogram of the scores should be identical or very close to each other for the synthetic images and real images. Typically, ROC curves are generated and AUC values are computed as part of these evaluations. Since the goal is to generate images that is inseparable from real ones, an AUC value of 0.5 is typically a good indicator of success.

In certain aspects, the present disclosure provides methods to convert synthetic MRI images to activity distributions and attenuation maps. In some of these embodiments, for example, GAN-based synthetic MRI images are converted to activity distributions and attenuation maps that can be used for PET or SPECT simulation. In certain of these embodiments, segmentation-based methods and/or CNN based methods disclosed herein are applied as part of these processes. In some embodiments, the activity concentration is based on measurements from PET or SPECT images. Optionally, attenuation maps are generated by filling each region of segmented synthetic MRI images with the corresponding attenuation coefficient. Regions-of-interest maps are also optionally generated from segmented synthetic MRI images. In addition, the results from these processes can used to train a CycleGAN as described further herein.

To illustrate, a cycle consistent generative adversarial network (CycleGAN) is optionally used to perform the image-to-image translation task of generating activity distributions and attenuation maps from synthetic MRI images. CycleGAN is an image-to-image translation method that does not need paired images for training. The CycleGAN typically consists of two generator networks and two discriminator networks as illustrated in, for example, FIG. 8. As shown in this exemplary embodiment, the first generator network (G1) learns to transform the MRI image to the activity distribution/attenuation map. The second generator (G2) learns the inverse transformation of the activity distribution/attenuation map back to the MRI image. The first discriminator network (D1) learns to discriminate the fake activity distributions/attenuation maps output by the first generator from real activity distributions/attenuation maps. As further shown, the second discriminator network (D2) learns to discriminate the real from fake MRI images. Real images are typically considered to be patient MRI images or synthetic MRI images. Training the generators and discriminators (G1 and D1; G2 and D2) follows a similar process as herein. In some embodiments, a feedforward fully-connected network architecture and a convolutional network architecture are used and compared with one another.

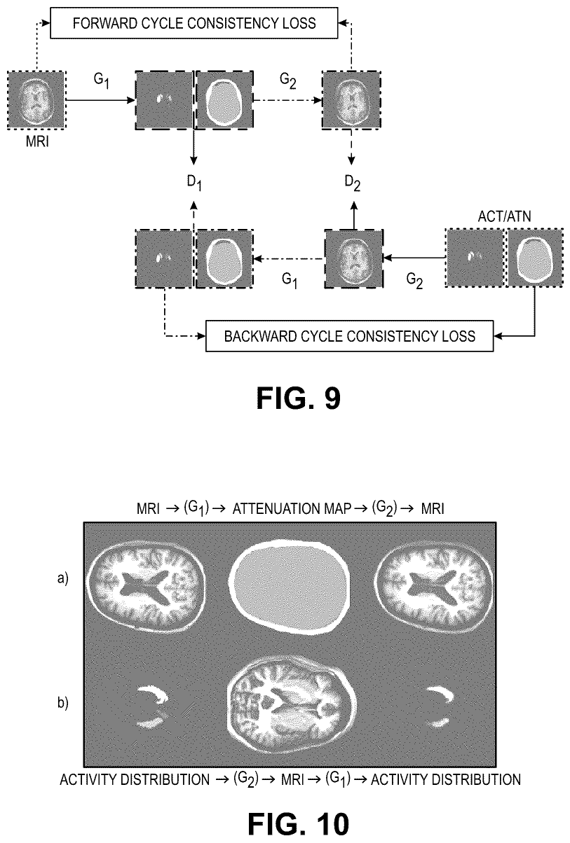

Unlike other image-to-image translation methods, CycleGAN does not require image pairs for training. A cycle consistency loss is used to enforce that the network learns the correct mapping from the MRI image to the activity distribution/attenuation map. In the cycle consistency loss (FIG. 9), the first generator G1 is given a real MRI image and produces an activity distribution/attenuation map. The generated activity distribution/attenuation map is then fed as an input to the second generator G2 which produces a fake MRI image (FIG. 10 (panel a)). This fake MRI image should correspond to the original real MRI image that is given as input to the first generator. To enforce this, the mean absolute error between the real and fake MRI image is typically computed in the cycle consistency loss. This is generally referred to as the forward cycle consistency loss. The backward cycle consistency loss is also accounted for where the second generator G2 is given a real activity distribution/attenuation map and produces a fake MRI image. This fake MRI image is given as input to the first generator G1 which produces a fake activity distribution/attenuation map (FIG. 10 (panel b)). The mean absolute error is computed between the real and fake activity distribution/attenuation map. This cycle consistency loss enforces a one-to-one mapping between the generated MRI images to the generated activity distributions/attenuation maps.

One advantage of CycleGAN is that it does not need a direct pairing between the MRI images and activity distributions/attenuation maps in the training set. This allow data to be augmented by adding more real patient MRI images that do not contain the corresponding activity distributions/attenuation maps from different sources to the training set. The training set can be further augmented by adding activity distributions and attenuation maps coming from different sources. For example, CT and transmission images from other data sources that do not have the corresponding MRI image can be used to augment the training set of attenuation images. Similarly, PET and SPECT images from other data sources that do not have the corresponding MRI image can be used to augment the training set of activity distributions. The realism of the synthetic MRI images produced by CycleGAN can be validated with existing MRI segmentation and analysis software, such as FreeSurfer, and with human observer studies as described further herein.

In some embodiments, the phantom population generated as described herein is validated. Unlike the validation methods of MRI images described above, there is no software or method available to validate a phantom population. Instead, the accuracy of the activity distributions and attenuation maps generated from the phantoms optionally includes performing Monte Carlo simulations. In some of these embodiments, simulated SPECT or PET data that mimic normal uptake or uptakes seen in, for example, Parkinson's disease is used. The SimSET-ARF method can be used for simulating SPECT data, and the OpenGATE can be used for simulating PET in some embodiments. The simulated data with clinical level of noise can then be reconstructed using same methods and parameters used in clinic in certain of these embodiments.

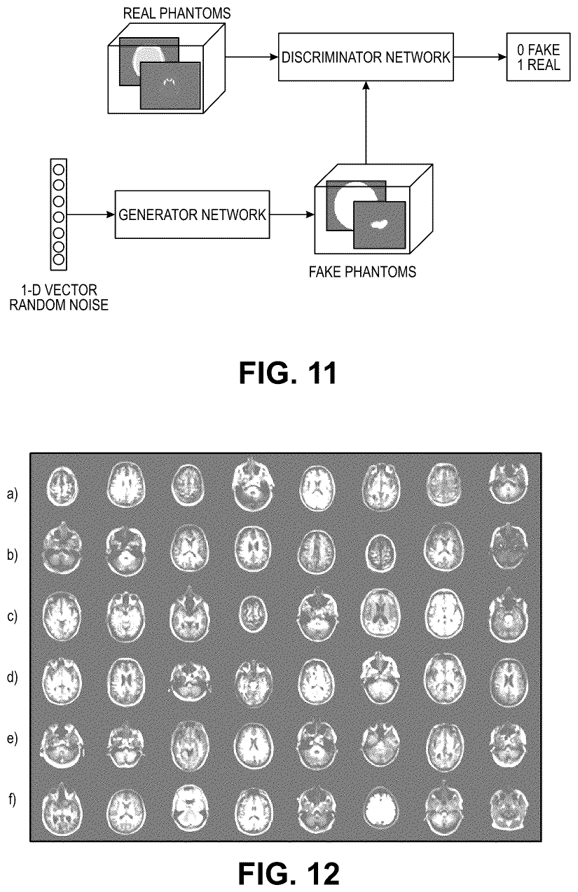

Some embodiments include using the resulting activity distributions and attenuation maps as training data to train a GAN-based approach to directly produce a population of generated synthetic activity distributions and attenuation maps without MRI images (FIG. 11). The GAN architectures and training procedures described herein can be used to directly generate synthetic activity distributions and attenuation maps. Additionally, the CycleGAN described herein can be trained to generate pairs of activity distributions and attenuation maps. Since CycleGAN can learn the one-to-one mapping between the activity distribution to the attenuation map and vice versa, the anatomical structure in the activity distribution and attenuation map pair can be preserved.

### Example: Generating Realistic Synthetic Brain MR Images with Generative Adversarial Networks

Introduction

In quantitative single photon emission computed tomography (SPECT) and positron emission tomography (PET) imaging, Monte Carlo simulation is an indispensable tool for the assessment of reconstruction algorithms, compensation methods, quantification and data analysis methods. In a simulation study truth is known and the image physics can be accurately modeled with Monte Carlo simulation, thus using simulated data can provide an accurate measurement of the accuracy and precision. To achieve this, a large set of digital phantoms that can realistically mimic the anatomy variations and tracer uptakes of human population is required. However, most of currently available digital phantoms, such as Zubal phantom and XCAT phantom, are usually developed based on one or averaged human data. They lack the ability to produce variations in the organ size and shape, and the body size and shape that can be seen in clinic.

One solution is to use a set of patient's anatomical imaging data, such as CT or MRI, to produce a phantom population. For example in brain SPECT and PET studies, MR images can be used to obtain anatomical information with high spatial resolution and high contrast for soft tissues. However, for task-based image quality assessment and AI-based research, thousands of data are often required. It is difficult to obtain such large amount of clinical MRI data. In addition, the time required for processing those data for phantom generation could be prohibitive.

Recently, deep learning approaches to generating simulated medical imaging data have shown promise. Specifically, it has been shown that generative adversarial networks (GANs) can generate clinically realistic MR images [Kazuhiro et al., “Generative Adversarial Networks for the Creation of Realistic Artificial Brain Magnetic Resonance Images,” Tomography, vol. 4, no. 4, p. 159 (2018)]. In this example, a GAN-based approach is used to generate realistic synthetic brain structural MR images that reflect the anatomical variability of the patient population. The approach is able to provide accurate 3D anatomical information required for generating realistic activity distributions needed for SPECT and PET simulations.

Methods

A. Brain MRI Data and Preprocessing

A total of 265 brain MR images of healthy controls and patients with various neurological diseases were used to train the proposed approach. A subset of this data was extracted from the open-source Parkinson's Progression Markers Initiative (PPMI) database. MR images were resampled to have a consistent voxel size of 1 mm³ and spatial dimensions of 256×256×256. The trans-axial cross-sections of these images were then down-sampled to have a voxel size of 4 mm² and spatial dimensions of 64×64. This yielded a dataset of 8,480 trans-axial cross-sections of brain MR images.

B. GAN Architecture

A deep generative adversarial network (GAN) architecture (FIG. 4) was developed to generate realistic synthetic brain MR images. The proposed GAN consisted of generator and discriminator networks. The generator is given a vector of Gaussian noise as input and generates synthetic brain MR images as output. The discriminator network is trained to distinguish the synthetic examples created by the generator from real brain MR images. The generator is then trained to create synthetic brain MR images that are realistic enough to fool the discriminator. The two networks are trained in this adversarial manner to generate realistic synthetic trans-axial cross-sections of brain MR images. The proposed network architecture is composed of strided convolutional layers and transposed convolutional layers that perform down-sampling and up-sampling in the generator and discriminator networks, respectively. Batch normalization is used throughout both the generator and discriminator networks to stabilize the adversarial learning process. Dropout with a drop probability of 0.5 is used in the last fully-connected layer of the discriminator before the classification of real and synthetic examples.

C. Training and Evaluation

The 265 brain MR images were used to train the GAN on the task of generating realistic synthetic MR images. The proposed method was visually evaluated and compared to real brain MR images from the clinical dataset. Representative examples were randomly selected from both the synthetic and real data distributions. The image intensity histograms of 8,500 synthetic images and 8,480 real images were also compared.

### RESULTS AND CONCLUSIONS

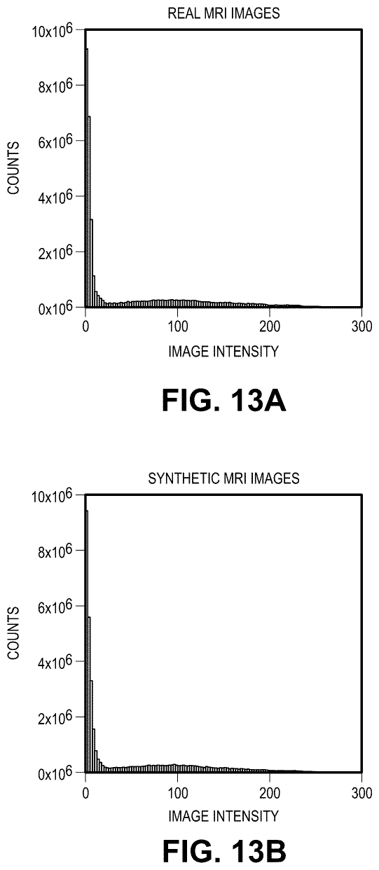

Representative examples of synthetic brain MR images generated by the proposed 3D GAN-based method are shown in FIGS. 12a-b and FIGS. 12d-e. The synthetic brain MR images are compared to real images in FIGS. 12c and f. Visually, the synthetic images generated by the GAN capture the variability in brain anatomy present in the real patient data. The intensity histogram of the synthetic images matched very closely to the intensity histogram of real images (FIG. 13). This provides further evidence that the GAN-based method provided realistic synthetic examples.

The proposed GAN-based approach showed significant promise for generating synthetic brain MR images. Future work will extend the proposed GAN-based approach to generate three-dimensional (3D) synthetic brain volumes of interest maps and activity distributions for SPECT and PET simulation.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. (canceled)

2. A method of conducting a medical imaging simulation, the method comprising:
training at least one generative adversarial network (GAN) with data from a plurality of synthetic magnetic resonance (MR) images and/or data from a plurality of synthetic computed tomography (CT) images to produce at least one activity distribution and/or at least one attenuation map; and,
using the activity distribution and/or the attenuation map to conduct the medical imaging simulation.

3.-6. (canceled)

7. The method of claim 2, comprising using the medical imaging simulations to perform at least one image-based task.

8. The method of claim 2, wherein the medical imaging simulations comprise single photon emission computed tomography (SPECT) and/or positron emission tomography (PET).

9.-11. (canceled)

12. The method of claim 2, wherein the plurality of synthetic MR images and/or the plurality of synthetic CT images comprises substantially accurate anatomical information.

13. The method of claim 2, wherein the data from the plurality of synthetic MR images and/or the data from the plurality of synthetic CT images comprises non-mean data.

14.-17. (canceled)

18. The method of claim 2, comprising using at least one compression technique to compress dimensionality of one or more of the plurality of synthetic MR images and/or the plurality of synthetic CT images.

19.-27. (canceled)

28. The method of claim 2, comprising using at least one convolutional neural network (CNN) and/or at least one cycle consistent GAN (CycleGAN) to produce the activity distribution and/or the attenuation map.

29. The method of claim 2, comprising validating anatomical realism and/or accuracy of one or more of the plurality of synthetic MR images, the plurality of synthetic CT images, the activity distribution, and/or the attenuation map.

30. The method of claim 2, comprising quantifying one or more activity distributions in one or more single photon emission computed tomography (SPECT) and/or positron emission tomography (PET) images to convert the plurality of synthetic MR images and/or the plurality of synthetic CT images to the activity distribution and/or the attenuation map.

31. The method of claim 2, comprising using the activity distribution and/or the attenuation map to train one or more GANs to produce additional activity distributions and/or additional attenuation maps.

32.-58. (canceled)

59. A system, comprising at least one controller that comprises, or is capable of accessing, computer readable media comprising non-transitory computer executable instructions which, when executed by at least one electronic processor, perform at least:
training at least one generative adversarial network (GAN) with data from a plurality of synthetic magnetic resonance (MR) images and/or data from a plurality of synthetic computed tomography (CT) images to produce at least one activity distribution and/or at least one attenuation map; and,
using the activity distribution and/or the attenuation map to conduct at least one medical imaging simulation.

60.-62. (canceled)

63. A computer readable media comprising non-transitory computer executable instruction which, when executed by at least electronic processor perform at least:
training at least one generative adversarial network (GAN) with data from a plurality of synthetic magnetic resonance (MR) images and/or data from a plurality of synthetic computed tomography (CT) images to produce at least one activity distribution and/or at least one attenuation map; and,
using the activity distribution and/or the attenuation map to conduct at least one medical imaging simulation.

64.-67. (canceled)

68. The system of claim 59, wherein the instructions further perform at least:
using the medical imaging simulations to perform at least one image-based task.

69. The system of claim 59, wherein the medical imaging simulations comprise single photon emission computed tomography (SPECT) and/or positron emission tomography (PET).

70.-72. (canceled)

73. The system of claim 59, wherein the plurality of synthetic MR images and/or the plurality of synthetic CT images comprises substantially accurate anatomical information.

74. The system of claim 59, wherein the data from the plurality of synthetic MR images and/or the data from the plurality of synthetic CT images comprises non-mean data.

75.-78. (canceled)

79. The system of claim 59, wherein the instructions further perform at least:
using at least one compression technique to compress dimensionality of one or more of the plurality of synthetic MR images and/or the plurality of synthetic CT images.

80.-88. (canceled)

89. The system of claim 59, wherein the instructions further perform at least:
using at least one convolutional neural network (CNN) and/or at least one cycle consistent GAN (CycleGAN) to produce the activity distribution and/or the attenuation map.

90. The system of claim 59, wherein the instructions further perform at least:
validating anatomical realism and/or accuracy of one or more of the plurality of synthetic MR images, the plurality of synthetic CT images, the activity distribution, and/or the attenuation map.

91. The system of claim 59, wherein the instructions further perform at least:
quantifying one or more activity distributions in one or more single photon emission computed tomography (SPECT) and/or positron emission tomography (PET) images to convert the plurality of synthetic MR images and/or the plurality of synthetic CT images to the activity distribution and/or the attenuation map.

92.-117. (canceled)
